(19) United States
(12) Patent Application Publication
(10) Pub. No.: US 2019/0015572 A1
(43) Pub. Date: Jan. 17, 2019

(54) UTERUS TO VENTRICULAR ASSIST DEVICE AND ARTIFICIAL HEART CONVERTER

(71) Applicant: KRISHNA PRASAD PANDURANGA REVANKAR, BANGALORE (IN)

(72) Inventor: KRISHNA PRASAD PANDURANGA REVANKAR, BANGALORE (IN)

(21) Appl. No.: 15/732,239

(22) PCT Filed: Apr. 10, 2016
(86) PCT No.: PCT/IN2016/000095

§ 371 Date: Sep. 17, 2018

(30) Foreign Application Priority Data
Apr. 10, 2015 (IN) 1886/CHE/2015

(51) Int. Cl. A61M 1/12; A61N 1/36; A61M 1/10

(52) U.S. Cl. CPC A61M 1/122; A61M 1/127; A61M 1/106; A61M 1/1003; A61N 1/36003

(57) ABSTRACT

A device to convert redundant uterus to ventricular assist device and total artificial heart by capturing and extracting muscular power of uterus being mechanical energy and transfer the energy to pump blood in the circulatory system with the transfer being one of indirect transfer through transfer fluid and where required directly to blood with the uterus based compression chambers directly pumping blood in the aortic circulation at least. A system of connecting joints and pipes organized around a mode change over valve set controlled by micro controller enabling the device to operate in different modes and an external pipe connector connected to external pump which greatly extends the utility of the invention enabling patient to treat problems from lung, kidney, liver, gastro intestinal system and manage sepsis. The purpose of the invention is give additional life span or time to a patient especially with heart failure.

### CLAIMS OF PRIORITY

This patent application claims priority from the Provisional Patent Application No. 1886/CHE/2015 filed on 10 Apr. 2015.

### FIELD OF TECHNOLOGY

This disclosure relates generally to technical fields of Bio medical devices in the area of ventricular support devices and artificial heart devices

### SUMMARY

The invention has been developed to address the problem of heart failure, which leads to progressive damage and ultimate failure. It is from the experience of the inventor that people with progressive worsening conditions like heart failure will greatly desire an extension of life as much as possible with the additional period being highly valuable. In order to meet this important need and give a continuation of life to the heart failure patients there is a need for artificial heart system since donor heart availability is dismally low. The pipe connector with external pump of the invention helps management of other organ problems such as those problems arising in kidney, lung and gastro intestinal track and also to manage sepsis. The efforts of various inventors as reflected in the prior art all have the problem of requiring a large power supply to pump blood. The present novel invention of using the redundant organ being uterus addresses the above problem in half the patients being women with a naturally given redundant organ with powerful muscles being the uterus which is not used after having two children as required to maintain the world population. Moreover spare uterus from donor can be transplanted to men if necessary to extend life.

There are a number of devices in the market and a number of prior art devices for ventricular support and total artificial heart. But they all have a problem of external power supply being electrical and pneumatic. The present invention chiefly lies in extracting energy from the uterus muscles and conveying it suitably to the circulatory system. Various attempts have been made to utilize muscles that can be spared, as can be seen in prior art patents U.S. Pat. No. 5,205,810, U.S. Pat. No. 5,205,810, U.S. Pat. No. 4,979,936, U.S. Pat. No. 5,984,857. U.S. Pat. No. 3,518,702 where muscles of the diaphragm have been used to provide support to the circulatory system but they all have problems of insufficient power, problem in synchronization, and practical adoption. The present invention overcomes this problem by the novel utilization of redundant uterus to supply the pumping energy by means of the energy capture and transfer device and method. The present invention supports the weak heart as a left ventricular assist device and adopts where required to replace the heart function thereby working as a total artificial heart. There are two mode of working either direct pumping of blood by compression chambers in uterus and by other mode being indirectly through transfer of pressure to blood using the pressure transfer fluid as intermediary to pick up pressure from uterus muscle contraction and then transfer the pressure to blood in the circulatory system.

The uterus is made to change from its resting state to become active by simulating pregnancy and its wall muscles trained to expand by hypertrophy under the influence of the hormone acting locally. Balloon is used to fit inside uterus during the enlargement process of simulated pregnancy with the balloon carrying the hormone dispenser.

In another aspect, the device comprises of making a new tubular connection with the major blood vessels namely aorta, vena cava, pulmonary artery and pulmonary veins by means of attaching a tube section containing valves with said valves determine mode of action of artificial heart device and said connection being a T joint with a additional flow channel. A bundle of connector pipes which terminate in a suitable pipe connector and the said valves having cable actuator, enables to change the natural hearts function of pumping blood by natural circulation to the blood circulation enabled by a artificial pump which is located in the abdominal cavity instead of being located in the thoracic cavity as in the prior art devices thereby by enabling various advantages like access to repair and easier installation of the artificial heart device.

The device basically comprises of pressure transfer system, blood collection system and blood pumping system. Pressure transfer fluid with storage of energy is used to operate total artificial heart of the type as available in prior art and commercially available wherein required application of pressure is from transfer fluid which extracts pressure energy from uterus and the transfer is controlled by the micro controller and its associated computer program instructions.

In yet another aspect, a second way of forming total artificial heart is described. When used as total artificial heart the compression chambers instead of pumping transfer fluid, are directly used to collect and pump blood in the pulmonary circulation and main aortic circulation, as there is no necessity for synchronization with natural heart. The electrical pulse to uterus wall muscles is controlled to give uniform and even compression and the lower beat frequency is compensated by larger discharge per beat. Similarly compression chambers using muscles of gastro intestinal system as power source, is also used to provide power to the left ventricular support device if not for total artificial heart.

Total artificial heart is formed with two or more sets of final blood pump to collect impure and deoxygenated blood and pumping it to lungs just like the function of right auricle and right ventricle being pumping blood to the pulmonary circulation and collecting the purified and oxygenated blood from pulmonary circulation to be pumped into the main circulatory system.

The device has a control circuits to manage time and frequency of pressure application to the circulating blood which is based on persons activity level and feed back from pulse oximetry sensors and other electrical voltage and pressure sensors forming part of the control unit which is made of a microprocessor with programmed instruction set and the microprocessor communicates with external computers by wireless link allowing monitoring. The microprocessor enables the configuration of the device to work in different modes of pumping being one of direct pumping of blood and by one of pumping through transfer fluid and by one of external pump based pumping, depending on the patients condition and need.

The above set of final blood pump is placed in the abdominal cavity rather than thorax for ease of placement and maintenance.

Connector pipe joints to main blood vessels with lateral pipes attached are grouped together and brought down through the diaphragm to connect to mode changeover valve set which is in turn connected to the blood pump and also connected to external connector which helps to make attachment to external pump. This piping system along with the external pump further enables the attachment of blood oxygenators, dialysis unit, sepsis management system, and drug and nutrient delivery unit, thereby creating a virtual intensive care unit on the move.

### BRIEF DESCRIPTION OF DRAWINGS

FIG. 1 Cross section view of compression chambers placed in the uterus.

FIG. 2 View of pressure storage tank with transfer fluid going to final blood pump and returning to receiving tank. Pressure storage tank having piston and energy storage spring.

FIG. 3A View of final blood pump with resilient member and FIG. 3B view of final blood pump without resilient member.

FIG. 4A View of external pressure pump for emergency use and for use during uterus growth under simulated pregnancy when hypertrophy of uterus muscles occurs with simulated pregnancy caused by hormones released locally.

FIG. 4B View of pipe connector of the external pump having external jacket and saline fluid inlet pipe which provides the saline fluid to surround the connecting pipes before making a connection in order to prevent entry of air bubbles.

FIG. 5 View of joint section with control valves and outlet and inlet pipe connected to aorta for use of the device as left ventricular assist device.

FIG. 6 View of device used in one embodiment for left ventricular assistance where blood from left ventricle is taken out through joint pipe from ventricle and given back to aorta after increasing pressure.

FIG. 7 View of the set of connector joints with T joint or Y joints grouped together and passed down as a bundle through diaphragm, having pipe connector, which is attached to the mode change over valve set.

FIG. 8 View of wrap on joint with T joint showing two edges brought together by insertion to surround a blood vessel.

FIG. 9 View of wrap on joint with T joint of FIG. 8 after joint extension and showing valve actuator cable which goes to the mode change over valve set and a parallel pipe to send flushing fluid when the pipe to which it is connected is not used for transferring blood.

FIG. 10 Cross section view of compression chambers placed in the large intestine.

FIG. 11 Schematic view of the entire device when placed in the body of a person taking the support of the device.

FIG. 12 Schematic view of the device with the external pump.

### DETAILED DESCRIPTION

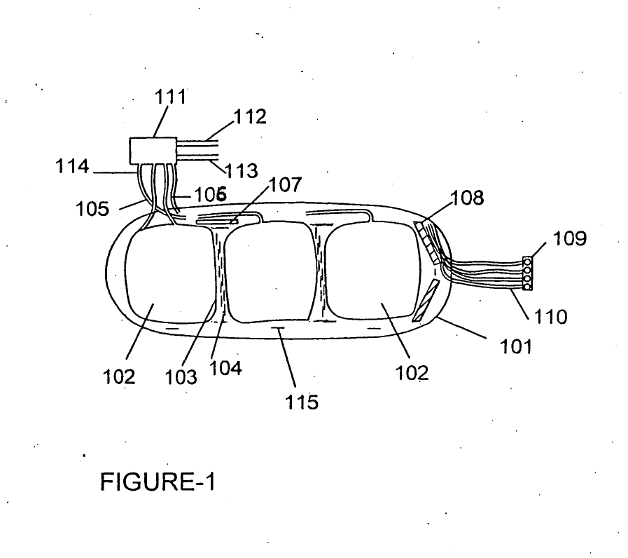

FIG. 1 Cross section view of compression chambers placed in the uterus 101, compression chamber 102, separator wall between chambers 103 having sliding elements 104 to enable flexibility in one direction only and being stiff in perpendicular direction, output pipeline 105, fluid return pipeline 106, output and return pipeline and other pipelines through erstwhile fallopian tube in one embodiment, sliding bar to hold resting chamber muscles well supported with rigid bar being 107, foldable insert which is a retainer and holder 108, being cup like with lock to fix the retainer rigidly after insertion into the uterus, thereby holding the compression chambers in the uterus after placement, external pipe connector 109 with pipes 110 which comes from the mode changeover valve set, passing through the uterus and then through cervix to out side the body which enables connection of the pipes of the external pump for use during development of uterus with muscle hypertrophy and for use in times of emergency and also facilitate other important life saving activity like oxygenation of blood, dialysis for kidney problem, dispense drugs and nutrients, and treat sepsis. Continuing the description of the drawing, mode changeover valve set 111 with solenoid actuators and electrical capacitor, battery and support electronic circuits, pipe for output of pressurized fluid 112, and pipe for return fluid 113, pipes from external pipe connector 114, pace maker being electrical contact 115 at the uterus wall.

The compression chamber is formed of flexible wall with material of the chamber being flexible elastic polymer, elastomer and one of rubber having a covering of biocompatible polymer. Output pipeline from compression chamber is taken out through one of cervix and one of erstwhile fallopian tube location. Only three chambers are shown in the drawing for sake of clarity.

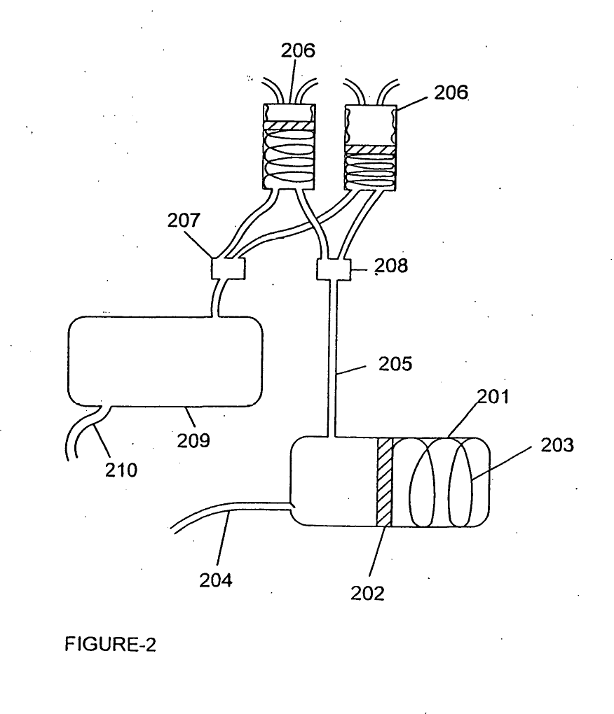

FIG. 2—system view of pressure storage tank with transfer fluid going to final blood pump and returning to receiving tank. Pressure storage tank 201, piston 202, energy storage spring 203, inlet from compression chambers 204, outlet to final blood pump 205, final blood pump 206, flow control valve 207 and 208 shown here separately for clarity in spite of being placed as valve group assembly, receiving tank 209, return line to compression chambers 210.

The power generated by the compression of the uterus wall is used to pump pressure transfer fluid, which transfers the pressure in the required manner and timing controlled by the control vales which are in turn actuated under the control of the microprocessor, the said transfer of pressure is to the blood taken from aorta through joint with outlet and inlet pipes and in case of a simpler embodiment wherein blood is taken from descending aorta portion in the abdomen and after pressurizing fed into the aorta near the heart.

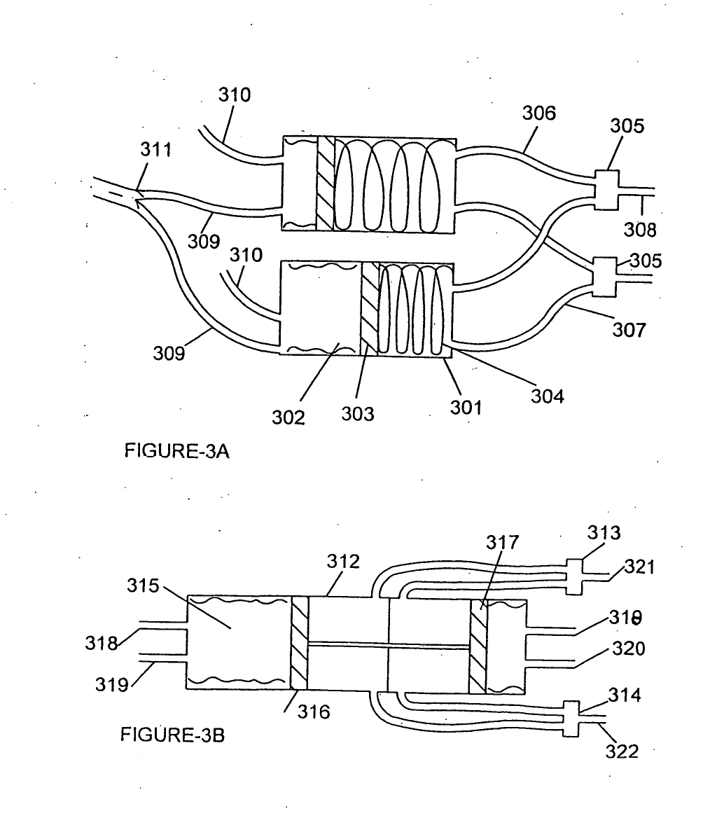

FIG. 3A View of final blood pump. Final blood pump 301, blood chamber being elastic and collapsible 302, piston being 303, resilient element being spring 304, control valve set 305 which makes one pump in suction mode and other in pumping mode, pressure transfer fluid input pipeline 306, pressure transfer fluid return pipeline 307, pressure transfer fluid input pipeline 308 connected to control valve set from pressure storage tank 201, blood input pipe 309 with directional valve 311 and pressurized blood output pipe 310.

FIG. 3B View of final blood pump without resilient element one part of the pump pressurizes blood while the other part of pump sucks in blood, final blood pump 312, control valve set 313 and 314 with pressure transfer fluid input pipe 321 and pressure transfer fluid return pipe 322 respectively, blood chamber being elastic and collapsible 315, piston being 316 and 317 and blood input pipe to the pumps being 318 and blood output pipe being 319 and 320.

As ventricular assist device is formed from two or more sets of final blood pump and an aorta based control valve and having pipe connections to the final blood pump for sending and then receiving high pressure blood from final blood pump. The two versions of final blood pump as shown in FIGS. 3A and 3B are used to ensure redundancy and as required. The same final blood pumps are used in the total artificial heart mode as required depending on the condition of the patient. The valves and pumps and storage unit are shown separately in drawing for sake of clarity and the same can be placed together as an integrated body to be implanted.

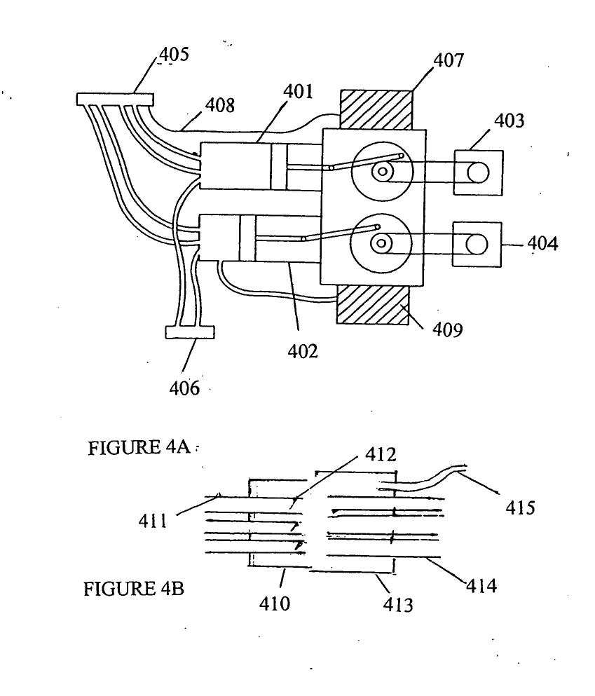

FIG. 4A view of external pressure pump for emergency use. pump 401 and 402 with piston, electric motor 403 and 404 driving the crankshaft of the pistons, female component of external pipe connector 405 which is a easy to connect, fast connector to connect to main device in the uterus, further present a pipe connector for connecting other devices like blood oxygenator, dialysis unit, drug and nutrient dispenser, sepsis management unit 406, battery and microprocessor unit 407 with electrical connector cable 408 to interact with microprocessor inside the body, reservoir tank for saline fluid to keep the pumps filled when not in used but ready to start immediately and the reservoir tank has a pipe connecting to the external connector outer cover to fill the space with saline fluid before making connection.

FIG. 4B view of the external connector with male socket 410 having cover and having pipes 411, with pipes having directional valve 412 as that connector is filled with saline fluid and kept ever ready for work, female socket 413 having cover and having pipes 414, and additionally has a pipe 415 to apply saline solution in the space of the connector socket before making the connection so that no air bubble gets into the circulation system.

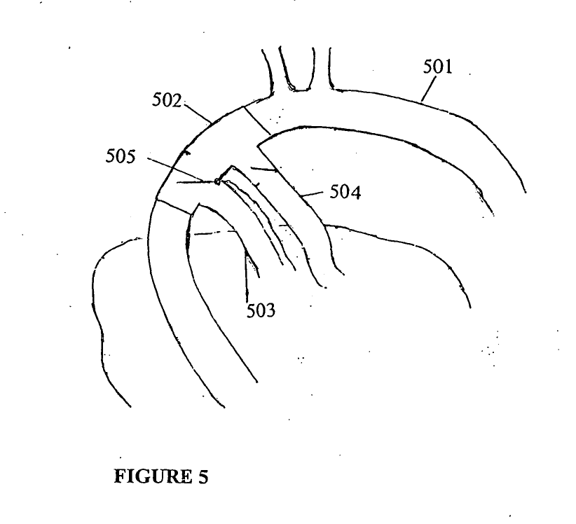

FIG. 5 View of connector pipe joint section with control valve attached in aorta 501, connector joint 502, outlet pipe 503 sending blood to final blood pump, inlet pipe 504 having directional valve, bringing in high pressure blood from final blood pump, control valve 505 actuated by cable running in a pipe parallel to outlet pipe and connected to mode change over valve set. The device works in the left ventricular assist mode to help the weak heart to recover strength.

Control valve fitted in aorta joint section allows diversion of weakly pumped blood from ventricle through the outlet pipe to the final blood pump for enhancing pressure and send the pressurized blood to the aorta trough the inlet pipe having directional valve.

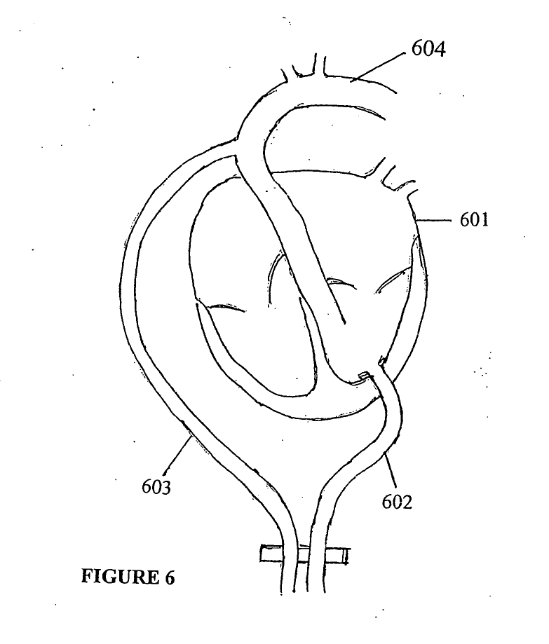

FIG. 6 View of device usage in one embodiment as left ventricular assistance device showing section view of heart 601, outlet pipe 602 from left ventricle through which flows a portion of oxygenated blood to final blood pump, input pipe 603 carrying high pressure blood output from final blood pump to aorta 604, in synchrony with left ventricular contraction. The pipes 602 and 603 are taken down through the diaphragm to the mode change over valve set which appropriately connects the flow to the requires final blood pump.

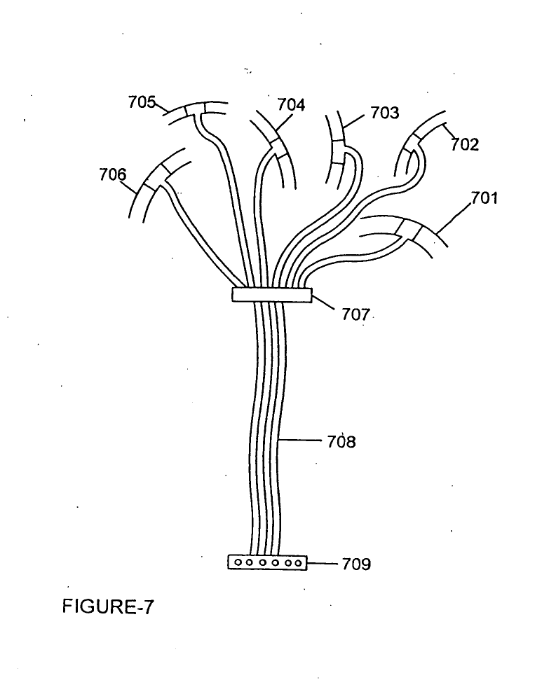

FIG. 7 View of a T joints attached to main blood vessels with output or input lateral pipes grouped together to form a bundle assembly 708, with pipe connector 707 and said bundle being passed down through a hole in the diaphragm to abdominal region and connected through the pipe connector 709 which is connected to the mode change over valve set which in turn selects connection to either final blood pump or to external pipe connector meant for attaching to external pump. The connector joint connected to aorta 701, connector joint connected to pulmonary veins 702 and 703, connector joint connected to pulmonary artery 704 and connector joint connected to superior and inferior venacava 705 and 706. There is also provided a pipe to vent excess pressure in the heart to prevent heart distension having valve controlled by the mode change over valve set. The T joint may also be a Y joint. The above pipe connector pipes may be connected directly to the main blood vessels in case there is low chance for heart to recover thereby avoiding the need for T joints.

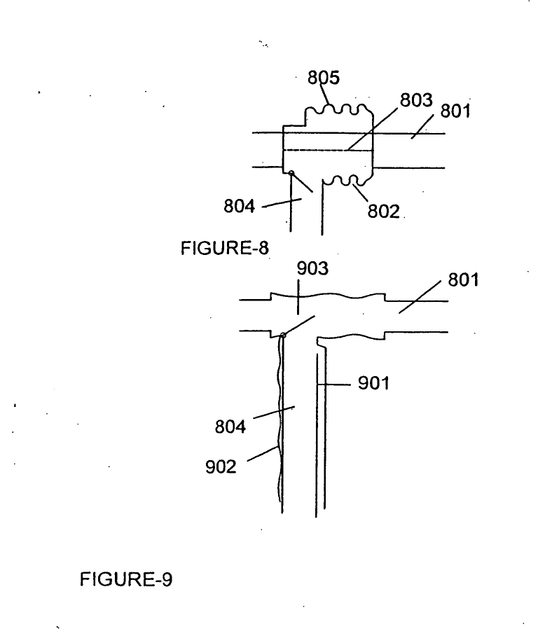

FIG. 8 view of wrap around T joint for connecting joint to blood vessel 801, wrap around T joint 802, cut edges 803 brought together after insertion around the blood vessel and joined by adhesive or stapling and then stapling or suturing the ends of the tube formed on wrapping, to the blood vessel, and further lateral pipe with valve 804, expandable bellow 805 to form a tube.

After joining the edges 803, a cutting tool which is operated by cable, is passed through the lateral pipe 804 and used to cut the blood vessel from inside and expand the bellow section to form connected tube. The purpose of this wrap around joint is to get a lateral connecting pipe without stopping the natural circulation in the said main blood vessels.

FIG. 9 view of the wrap around T joint as in FIG. 8 after internal cut of the blood vessel and extension of the joint showing blood vessel 801, lateral pipe 804 having valve 903, with the valve being actuated by cable 902 which is connected to mode change over valve set for actuation, parallel pipe 901 to send flushing fluid when the pipe to which it is connected is not used for transferring blood.

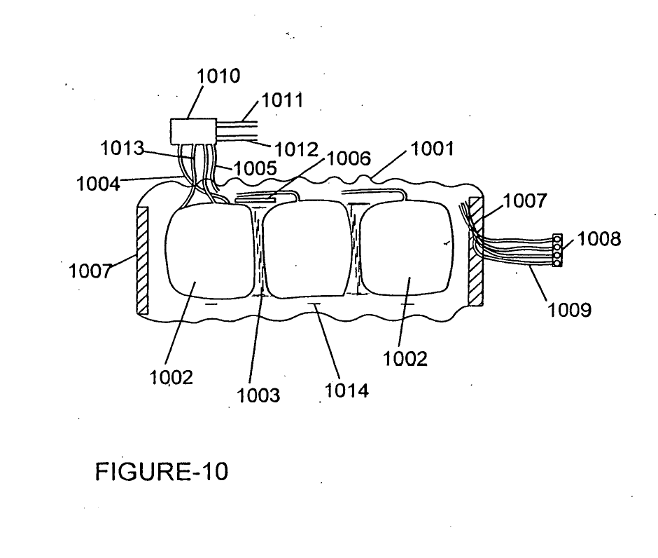

FIG. 10 cross section view of compression chambers placed in the spare large intestine portion being 1001, compression chamber 1002, separator wall between chambers 1003 having sliding elements to enable flexibility in one direction only and stiff in perpendicular direction, output pipeline 1004, fluid return pipeline 1005, sliding bar to hold resting chamber muscles well supported with rigid bar being 1006, retainer and holder 1007 connected at end, pipe connector 1008 with pipes 1009 which comes from the mode changeover valve set and taken out side the body which enables connection of the pipes of the external pump during training period to bring about muscle hypertrophy and for use in times of emergency and also facilitate other important life saving activity like oxygenation of blood, dialysis for kidney problem, dispense drugs and nutrients, and treat sepsis. Continuing the description of the drawing, mode changeover valve set 1010 with solenoid actuators and electrical capacitor, battery and support electronic circuits, pipe for output of pressurized fluid 1011, and pipe for return fluid 1012, pipes from external pipe connector 1013, pace maker electrical contact 1014

The compression chamber is designed where required to be fitted into section of large intestine cut and separated and, with cut ends of digestive track and anus connected intestinal end joined surgically and the resulting spare portion of large intestine is used for pumping the pressure transfer fluid to supply power in left ventricular assist mode. Similarly a portion of the stomach which can be spared by stapling and suturing is used as power source for pumping the pressure transfer fluid with all other component and usage as described for uterus based system work similarly in this above two source of power.

In the drawings described, only three compression chamber are shown for sake of clarity and ease of understanding in spite of more number of chambers being used in the device.

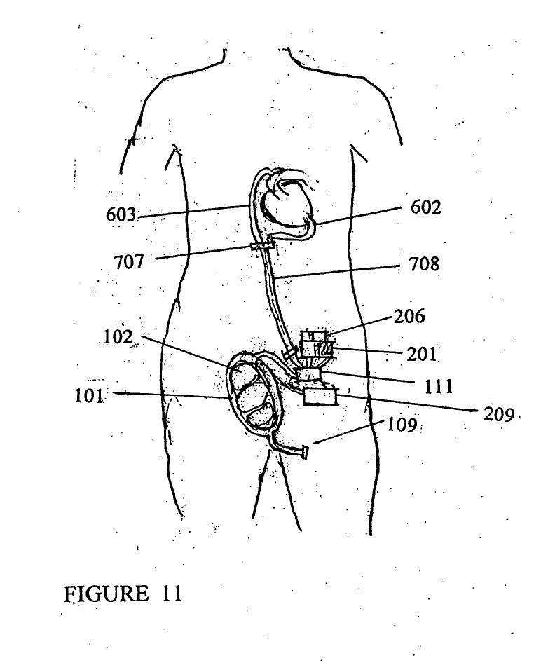

FIG. 11 a schematic view of the entire device as placed in a human body to do the job of the heart. Shown in the figure are left ventricular assistance device showing sectional view of heart with outlet pipe 602 from left ventricle, input pipe 603 connected to aorta, connector 707, bundle of pipes 708 coming down to the abdominal region, pressure storage tank 201, final blood pump 206, receiving tank 209. The figure also shows compression chambers placed in the uterus 101, compression chamber 102, external pipe connector 109 attached to pipes that come from the mode changeover valve set 111, with said pipes coming out after passing through the uterus. The elements 111, 201, 206, and 209 have been shown as separate elements only for the sake of clarity while in use they are integrated to form a single compact unit.

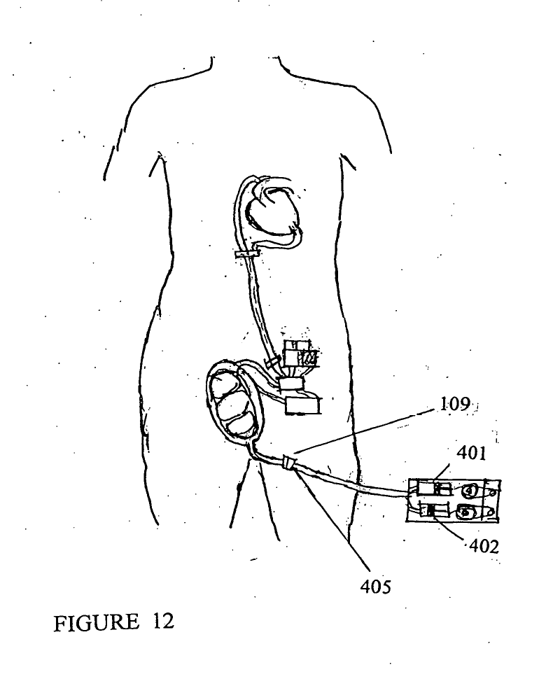

FIG. 12 a schematic view of the entire device with external pressure pump for emergency use with pump 401 and 402 with piston and electric motor, external pipe connector 405 which is a easy to connect being a fast connector meant for connecting to pipe connector 109. External pressure pump is important for providing extra circulation of blood needed to nourish the uterus when the uterus muscles are enlarging by hypertrophy so that the already weak heart is not over loaded.

The different modes of working of the system namely ventricular support mode, total heart replacement mode and external circulation support mode and the two different ways of pressurizing blood one being indirect through an intermediary transfer fluid and other mode by means of direct pumping of blood by the compression chambers, may appear complicated however the problem of heart failure is highly complicated and complex and hence need an elaborate and challenging system to solve and extent life in the last period and phase of the life of a person. The change of modes is facilitated by the mode change over valve set which is in turn controlled by the micro controller.

Although the present embodiments have been described with reference to specific example embodiments, it will be evident that various modifications and changes may be made to these embodiments without departing from the broader spirit and scope of the various embodiments.

In addition, it will be appreciated that the various operations, processes, and methods disclosed herein may be embodied in a machine-readable medium and/or a machine accessible medium compatible with a data processing system (e.g., a computer system), and may be performed in any order (e.g., including using means for achieving the various operations). Accordingly, the specification and drawings are to be regarded in an illustrative rather than a restrictive sense.

I claim:

1) A artificial heart device powered by a redundant organ being uterus of a human body with power being the muscular power of uterus with said device comprising of:
a plurality of compression chambers with inlet and outlet pipes for fluid which is pressurized and said pipes having valves;
a electrical pacing means to actuate the required muscles of the uterus wall;
a fluid pressure transfer and pressure storage means;
a hormone supply and release means;
a plurality of connecting tubes with lateral joints and connectors;
a collection of valves with actuators named hereafter as mode change over valve set;
a micro controller to control actuation of valves, control the said pacing means, take sensor input signal and also provide emergency alert.

2) The device as claimed in claim 1 wherein a plurality of compression chambers being made from one of elastomeric material and the compression chambers being compressed turn by turn and wherein the compression chambers having walls adjacent to each chamber provided with sliding rigid elements embedded in such away that the wall is compressible in one direction but rigid and stiff in perpendicular direction and wherein the chambers have directional valves to allow fluid to exit on compression and enter in on relaxation.

3) The device as claimed in claim 1 wherein a pressure transfer chamber has resilient member being one of a spring to store the pressure energy and valves whose actuation and timing are being controlled to transfer the fluid pressure to final blood pump in a manner to pump blood with timing which is synchronous to the natural heart beat and wherein pressure transfer fluid is used to operate where necessary other artificial heart devices such as those already in prior art and those commercialized.

4) The device as claimed in claim 1 wherein a pair or multiple pairs of final blood pump having piston moved by pressure transfer fluid whose timing of movement is controlled by control valve and the said final blood pump receives blood from one of ventricular outlet pipe and from one of outlet pipe from connector joint connected to the aorta and from one of mode change over valve set and then, after pressurizing the blood discharges the pressurized blood into the aorta for circulation in synchronization with the weak pumping heart when device is used as left ventricular assist device and when device is used as total artificial heart, discharge the pressurized blood in aortic circulation and pulmonary circulation.

5) The device as claimed in claim 1 wherein a fluid return chamber collects the returning fluid from final blood pumps and then fills the multiple compression chambers and it also holds some amount of reserve of transfer fluid and wherein additional fluid return chambers are used to collect and hold blood when device is used in another mode being as total artificial heart, pumping blood directly from the multiple compression chambers into the circulatory system.

6) The device as claimed in claim 1 wherein an external pump driven by an electric motor and having battery, transfer fluid reservoir and micro processor communicating with the microprocessor in the body through cable which comes along with external pipe connector with the said external pump takes over pumping pressure transfer fluid during initial growth and training period of the uterus muscles to bring about hypertrophy of the muscles and during emergency arising due to insufficient pumping and at times when maintenance work is needed and

7) The device as claimed in claim 1 wherein the external pump has a fast pipe connector with socket which is enclosed by saline fluid before making connection to prevent air bubble entry and with the said connector enabling a patient to connect the main artificial heart device with the external pump and wherein external pump has another extra connector which enables along with mode change over valve set to pump blood to circulate in one of a blood oxygenator, a blood dialysis unit, a drug and a nutrient dispenser, and a sepsis management unit thereby providing artificial support for other organs like lung, kidney, liver and gastro intestinal system when needed.

8) The device as claimed in claim 1 wherein a plurality of connecting tubes with joints being one of T joints and one of Y joints with lateral tubes and having directional valves and valves with actuating cable wherein the cable is actuated by mode change over valve set, with the said joints being connected to main blood vessels namely aorta, superior and inferior vena cava, pulmonary vein and pulmonary artery and with the lateral tubes grouped together and passed down along with electrical cables from sensors through a hole made in the diaphragm and brought to the abdominal region to connect to the mode change over valve set which in turn connects the tubes to the uterus pump and also enables to connect with the external pump through external pipe connector which passes out through the cervix and wherein the above said connecting tubes is connected directly to the said main blood vessels without the said connector joints in patients having heart with low recovery chances where the ventricular assist mode is not useful.

9) The connecting tube with joints as claimed in claim 8 wherein the joint is one of a wrap on connector joints with a central cut in the wrapping portion of the joint to enable its insertion over, that is to surround a main blood vessel, with cut edges joined after insertion by one of using adhesive and one of staple and the tube so formed is connected to the blood vessel by stapling or by sutures with the joint having lateral pipes with valve and actuating cable and a parallel flushing fluid inlet pipe and then after the procedure of wrapping on the joint and attaching to the blood vessel, the blood vessel is cut from inside using a tool with cutter inserted through the lateral pipe and operated by cable and then extending the bellow portion of the joint.

10) The device as claimed in claim 1 wherein a micro controller unit with programmed instructions to actuate muscle by electrical pulsing for pacing the required muscle at required time and allowing rest period, actuate valve opening by giving actuating signal to the mode change over vale set based solenoid actuators, sense pressure from pressure sensors and oxygen saturation information from pulse oximetry sensor, synchronies the working of final heart pump with natural heart beat and also control pumping activity of the device when used as total artificial heart and forecast emergency situation based on sensor data and give a warning alarm by actuating a buzzer and vibrator to alert the patient to connect to external pump for emergency support and wherein the micro processor communicates with microprocessor present in the external pump and other external computers by wireless means.

11) The device as claimed in claim 1 wherein a mode change over valve set being a collection of valves and tubes to enable various flow configurations as required thereby enabling required changes in the mode of operation from ventricular assist mode to total artificial heart mode and to external pump mode when required and wherein the mode change over valve set unit has electrical capacitor, battery, electronic circuits and solenoid for actuating the valves as required and wherein the mode changeover valve set has clot scavenging filters unit.

12) The device as claimed in claim 1 wherein a hormone release system for releasing in the uterus, locally acting hormones to simulate pregnancy and maintain the uterus muscle in functional state continuously.

13) The device as claimed in claim 1 wherein a foldable insert to act as retainer and holder which is locked to fixed position after insertion behind the multiple compression chambers wherein the said insert is being placed near the cervix to provide proper grip to the pumping device in the uterus and prevent its exit from the uterus.

14) The device as claimed in claim 1 wherein some of the multiple compression chambers are used to pump blood directly into the aortic circulation instead of indirect way through the transfer fluid for getting better pumping efficiency when the device is used as total artificial heart and wherein some of the other compression chambers are used to pump pressure transfer fluid for use in pumping blood in pulmonary circulation as required and wherein the mode change over valve set controls the selection of flow pipes to connect to required pumps.

15) The device as claimed in claim 1 wherein part of the energy of the uterus muscles is converted into electrical energy by generator formed of magnetic element located in spacer adjacent to wall of multiple compression chamber which moves on compression in a manner relative to fixed stator coils which generates electricity to power the microprocessor and valve actuation solenoids.

16) A artificial heart device powered by redundant muscles of the human body such as muscular power of muscles from gastro intestinal system from being one of spare area of large intestine and spare area of stomach after stapling a certain portion of stomach with said device comprising of:
a plurality of compression chambers with inlet and outlet pipes for fluid which is pressurized and said pipes having valves.
An electrical pacing means to actuate the required muscles;
a fluid pressure transfer and pressure storage means;
a plurality of connecting tubes with lateral joints and connectors;
a collection of valves with actuators named hereafter as mode change over valve set;
a micro controller to control actuation of valves, control the said pacing means, take sensor input signal and also provide emergency alert.
